(19) United States
(12) Patent Application Publication
(10) Pub. No.: US 2022/0211768 A1
(43) Pub. Date: Jul. 7, 2022

(54) CONDITIONED MEDIUM AND METHODS OF USE THEREOF

(71) Applicant: Histogen, Inc., San Diego, CA (US)

(72) Inventors: Gail K. Naughton, San Diego, CA (US); Martin Latterich, San Diego, CA (US)

(21) Appl. No.: 17/607,842

(22) PCT Filed: Apr. 30, 2020
(86) PCT No.: PCT/US2020/030828

§ 371 Date: Oct. 29, 2021

Related U.S. Application Data
(60) Provisional application No. 62/842,245, filed on May 2, 2019.

(51) Int. Cl. A61K 35/33; A61K 8/98; C12N 5/077; A61Q 19/08; A61P 17/18

(52) U.S. Cl. CPC A61K 35/33; A61K 8/981; C12N 2500/02; A61Q 19/08; A61P 17/18; C12N 5/0656

(57) ABSTRACT

The present invention is directed to a methods for the promotion of autophagy and/or proteasome activation in a subject by administering a cell culture conditioned media (CCM) composition. The present invention also provides methods for reducing symptoms of aging and treating diseases or conditions including Alzheimer's disease and lung fibrosis.

### CROSS-REFERENCE TO RELATED APPLICATIONS

This application claims benefit of priority under 35 U.S.C. § 119(e) of U.S. Ser. No. 62/842,245, filed May 2, 2019, the entire contents of which is incorporated herein by reference in its entirety.

### BACKGROUND OF THE INVENTION

### Field of the Invention

The present invention relates generally to the production and use of growth factors and/or conditioned culture medium compositions and more specifically to compositions to treat conditions and disorders.

### Background Information

The extracellular matrix (ECM) is a complex structural entity surrounding and supporting cells that are found in vivo within mammalian tissues. The ECM is often referred to as the connective tissue. The ECM is primarily composed of three major classes of biomolecules including structural proteins such as collagens and elastins, specialized proteins such as fibrillins, fibronectins, and laminins, and proteoglycans. Conditioned culture medium (CCM) contains biologically active components obtained from previously cultured cells or tissues that have released into the media substances affecting certain cell function. It has been found that ECM and CCM compositions derived in vitro from cells grown under hypoxic or normoxic conditions have therapeutic properties beneficial for treating certain conditions.

Growth of ECM and CCM compositions in vitro and their use in a variety of therapeutic and medical applications have been described in the art. One therapeutic application of such ECM and CCM compositions includes the promotion of autophagy and/or proteasome activation.

Autophagy is a conserved intracellular catabolic process that is responsible for the targeting of proteins, organelles, exogenous particles and microorganisms to the lysosome for their subsequent degradation. Autophagy is also globally involved in a number of key physiological processes including tumorigenesis, the immune response, and apoptosis and disordered autophagy has been associated with a variety of diseases including infectious diseases and cancers and cardiomyopathies. Autophagy is a stress response that becomes enhanced when nutrients and growth factors are limited and in cases when macromolecules are damaged, fibrillated, and aggregated. Intrinsic and extrinsic factors that cause aging result in the accumulation of cellular damage which includes mitochondrial damage, reduced efficiency of energy production, and protein aggregation. Treatment of young dermal fibroblasts with inhibitors of lysosomal protease in order to mimic aged fibroblasts with reduced autophagic activity resulted in an altered content of procollagen, elastin, hyaluronan, and the breakdown of collagen fibrils. It is evident that a loss in autophagy activity associated with the aging process leads to the deterioration of dermal integrity and skin fragility. Proteostasis involves a variety of mechanisms responsible for the stabilization of correctly folded proteins and the degradation and removal of misfolded polypeptides through lysosome or proteasome activity. In addition, many studies have shown that aging alters proteostatis and is associated with accumulated cellular debris and cell damage. Several studies in various human tissues have shown that proteasome function and activity decline during aging and studies in healthy centenarians have revealed that their proteasomes maintain their function. The finding that in the normal course of aging tissues and organs begin to lose their ability to effectively regenerate has been attributed to stem cells in the tissue losing their “stemness” which is known as stem cell exhaustion. In vitro studies have demonstrated that proteostasis slowdown associated with senescence negatively affects stemness of hMSCs and such loss of proliferation capacity and stemness can be reversed with proteasome activation. A variety of in vitro and in vivo studies in knockout mice, aged human fibroblasts, and stem cells has provided further evidence that reversing proteostasis slowdown also leads to the retardation of senescence. It has been hypothesized that stem cell exhaustion is a major factor in tissue aging and studies have supported that stem cell rejuvenation may reverse aging. Recent studies have demonstrated that stem cell depletion and diminished regenerative capacity is linked to increased FGF2. The administration of exogenous growth factors by injection of stem cells or growth factors from young animals has been demonstrated to delay and reverse tissue degenerative changes. Transplantation of muscle stem cells from young mice into progeroid mice expanded the lifespan of the aged animals and reversed aging processes even in organs where donor cells were not detected, supporting the hypothesis that secreted factors from the injected cells positively affected cells and tissues throughout the body.

The aging process results in decreased growth factor and cytokine production and subsequent decrease in cell-cell communication and optimal tissue function. The accumulation of cell debris and reduction of immune regulation due to an aging stem cell population in the bone marrow, result in increased tissue inflammation in the skin and other organs. These age-related changes underscore the importance of restoring normal skin function by reestablishing intracellular signaling, dermal fibroblast precursor activation, in addition to normal autophagy and proteasome activity. The skin is the largest organ of the body with complex functions and a high regenerative capacity. It is highly prone to injury when exposed to injury by UV radiation, pollution and other harmful agents. A variety of stem cell populations exist in the skin including follicular and interfollicular stem cells, dermal mesenchymal stem cells, endothelial and hematopoetic stem cells, and hair follicle stem cells. Skin derived precursors (SKPs) are located in the dermis and integral to dermal reconstruction. Studies have demonstrated the ability of MrCx to stimulate at least two skin precursor cells, hair follicle stem cells in the scalp to support the growth of new hair in humans and dermal precursors to help produce new dermal fibroblasts.

Proteasomes are protein complexes which degrade unneeded or damaged proteins, marked by ubiquitylation, by proteolysis, a chemical reaction that breaks peptide bonds. Proteasomes are part of a major mechanism by which cells regulate the concentration of particular proteins and degrade misfolded proteins. Proteins are tagged for degradation with a small protein called ubiquitin. The tagging reaction is catalyzed by enzymes called ubiquitin ligases. Once a protein is tagged with a single ubiquitin molecule, this is a signal to specific outer chain ligases to attach additional ubiquitin molecules. The result is a polyubiquitin chain that is recognized by cap structures of the proteasome, allowing the central proteasome core to degrade the tagged protein. The degradation process yields peptides of about seven to eight amino acids long, which can then be further degraded into shorter peptides and amino acids which can be used in synthesizing new proteins.

Regulation of the autophagy and proteasome activation pathways may be useful for the treatment of various disease and disorders or for the reduction in the symptoms of aging.

### SUMMARY OF THE INVENTION

The present invention is based in part on the seminal discovery that cells cultured on surfaces (e.g., in monolayers or layers on one-dimensional surfaces; two-dimensional or three-dimensional surfaces) produce ECM compositions and CCM compositions. The ECM and CCM compositions produced by culturing cells under normal, or normoxic, or under hypoxic conditions containing one or more embryonic proteins have a variety of beneficial applications.

In one embodiment, the present invention provides a method of promoting autophagy and/or proteasome activation in a subject comprising administering to the subject a cell culture conditioned medium (CCM) composition. In one aspect, the CCM composition is produced by culturing cells in a suitable growth medium, wherein the cells produce and secrete a cell culture conditioned medium (CCM) composition. In another aspect, the cells are cultured under hypoxic or normoxic conditions. In certain aspects, the hypoxic conditions comprise 1-5% O₂. In an additional aspect, the cells are fibroblast cells and the cells are grown under one, two or three dimensional conditions. In a further aspect, the cells are grown in a monolayer, on beads or on mesh. In one aspect, the CCM composition is administered by topical, oral or intravenous administration. In one aspect, the expression of autophagy and/or proteasome associated polypeptides Autophagy related 5, Autophagy related 7, Autophagy related 12, Beclin-1, Microtubule-associated proteins 1A/1B light chain 3, Proteasome maturation protein, Proteasome subunit beta type-5, Proteasome subunit beta type-5 or a combination thereof is increased following administration of the CCM. In one aspect, the expression of the autophagy and/or proteasome related genes AGT5, ATG7, ATG12, BECN1, MAP1LC3, POMP, PSMB5, PSMB6 or a combination thereof is increased following administration of the CCM. In one aspect, the CCM composition is administered in a nano-container conjugated to a targeting moiety for specific tissue delivery. In specific aspects, the targeting moiety for specific tissue delivery is an aptamer, antibody, antibody fragment, peptide or affinity ligand that recognizes a tissue specific surface or internalization biomarker.

In an additional embodiment, the present invention provides a method of reducing symptoms of aging in a subject comprising administering to the subject a cell culture conditioned medium (CCM) composition. In one aspect, the CCM composition is produced by culturing cells in a suitable growth medium, wherein the cells produce and secrete a cell culture conditioned medium (CCM) composition. In an additional aspect, the symptoms of aging are selected from the group consisting of fine lines, wrinkles, dark spots, dry skin, loosening of the skin and dulling of skin tone. In a further aspect, the CCM composition is administered topically.

In a further aspect, the present invention provides a method of preventing, treating and/or ameliorating symptoms of a disease or condition in a subject comprising administering to the subject a cell culture conditioned medium (CCM) composition. In one aspect, the CCM composition is produced by culturing cells in a suitable growth medium, wherein the cells produce and secrete a cell culture conditioned medium (CCM) composition. In another aspect, the disease or condition is Alzheimer's disease, lung fibrosis, Huntington's disease, Amyotrophic lateral sclerosis, muscular dystrophies characterized by downregulated proteasome functions and other diseases of aging caused by diminished autophagy and proteasome activity. In a further aspect, the CCM composition is administered by topical, oral, nasal, alveolar lavage, inhalation, intravenous or intracranial administration.

### DESCRIPTION OF THE FIGURES

FIG. 1 shows the expression levels of proteasome related genes POMP, PSMB5 and PSMB6 in human 3D skin models exposed to UV light and treated or untreated with a CCM composition.

FIG. 2 shows the expression levels of autophagy related genes ATG5, ATG7, ATG12, BECN1 and MAP1LC3 in human 3D skin models exposed to UV light and treated or untreated with a CCM composition.

### DETAILED DESCRIPTION OF THE INVENTION

The present invention relates to a method for making and using growth factor compositions, including but not limited to culture conditioned media (CCM). In particular the compositions are generated by culturing cells (e.g., fibroblasts) under culture conditions on a surface (e.g., one-dimensional, two-dimensional or three-dimensional) in a suitable growth medium producing an extracellular matrix (ECM) and CCM composition. Both ECM and CCM fractions may be used separately or in combination for a variety of applications.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

In one embodiment, the present invention provides a method of promoting autophagy and/or proteasome activation in a subject comprising administering to the subject a cell culture conditioned medium (CCM) composition. In one aspect, the CCM composition is produced by culturing cells in a suitable growth medium, wherein the cells produce and secrete a cell culture conditioned medium (CCM) composition. In another aspect, the cells are cultured under hypoxic or normoxic conditions. In certain aspects, the hypoxic conditions comprise 1-5% O₂. In an additional aspect, the cells are fibroblast cells and the cells are grown under one, two or three dimensional conditions. In a further aspect, the cells are grown in a monolayer, on beads or on mesh. In one aspect, the CCM composition is administered by topical, oral or intravenous administration. In one aspect, the expression of autophagy and/or proteasome associated polypeptides Autophagy related 5, Autophagy related 7, Autophagy related 12, Beclin-1, Microtubule-associated proteins 1A/1B light chain 3, Proteasome maturation protein, Proteasome subunit beta type-5, Proteasome subunit beta type-5 or a combination thereof is increased following administration of the CCM. In one aspect, the expression of the autophagy and/or proteasome related genes AGT5, ATG7, ATG12, BECN1, MAP1LC3, POMP, PSMB5, PSMB6 or a combination thereof is increased following administration of the CCM. In one aspect, the CCM composition is administered in a nano-container conjugated to a targeting moiety for specific tissue delivery. In specific aspects, the targeting moiety for specific tissue delivery is an aptamer, antibody, antibody fragment, peptide or affinity ligand that recognizes a tissue specific surface or internalization biomarker.

One example of culturing cells includes culturing fibroblast cells under normoxic or hypoxic conditions of about 1-5% oxygen on microcarrier beads or a three dimensional surface in a suitable growth medium, for at least 2 weeks, thereby producing multipotent stem cells, wherein the multipotent stem cells produce and secrete into the growth medium the composition. This method provides both a non-soluble extracellular matrix (ECM) composition and a soluble CCM composition. The non-soluble composition includes those secreted ECM proteins and biological components that are deposited on the support or scaffold. The soluble composition includes culture media or conditioned media in which cells have been cultured and into which the cells have secreted active agent(s) and includes those proteins and biological components not deposited on the scaffold. Both compositions may be collected, and optionally further processed, and used individually or in combination in a variety of applications as described herein.

Culture medium compositions typically include essential amino acids, salts, vitamins, minerals, trace metals, sugars, lipids and nucleosides. Cell culture medium attempts to supply the components necessary to meet the nutritional needs required to grow cells in a controlled, artificial and in vitro environment. Nutrient formulations, pH, and osmolarity vary in accordance with parameters such as cell type, cell density, and the culture system employed. Many cell culture medium formulations are documented in the literature and a number of media are commercially available. Once the culture medium is incubated with cells, it is known to those skilled in the art as “spent” or “conditioned medium”. Conditioned medium contains many of the original components of the medium, as well as a variety of cellular metabolites and secreted proteins, including, for example, biologically active growth factors, inflammatory mediators and other extracellular proteins. Cell lines grown as a monolayer or on beads, as opposed to cells grown in three-dimensions, lack the cell-cell and cell-matrix interactions characteristic of whole tissue in vivo. Consequently, such cells secrete a variety of cellular metabolites although they do not necessarily secrete these metabolites and secreted proteins at levels that approach physiological levels. Therefore the composition of a CCM is dependent on the cell culture conditions such as growing the cells on a one, two or three dimensional surface and whether cells are grown under normoxic or hypoxic conditions.

The secretion of extracellular proteins into conditioned cell media such as growth factors, cytokines, and stress proteins opens new possibilities in the preparation of products for use in a large variety of areas including tissue repair, e.g., in the treatment of wounds and other tissue defects such as cosmetic defects as well as human and animal feed supplements. For example, growth factors are known to play an important role in the wound healing process. In general, it is thought desirable in the treatment of wounds to enhance the supply of growth factors by direct addition of these factors.

The cultivation materials providing three-dimensional architectures are referred to as scaffolds. Spaces for deposition of ECM are in the form of openings within, for example woven mesh or interstitial spaces created in a compacted configuration of spherical beads, called microcarriers.

The methods described herein provide both a non-soluble extracellular matrix (ECM) composition and a soluble CCM composition. The non-soluble composition includes those secreted ECM proteins and biological components that are deposited on the support or scaffold. The soluble composition includes culture media or conditioned media in which cells have been cultured and into which the cells have secreted active agent(s) and includes those proteins and biological components not deposited on the scaffold. Both compositions may be collected, and optionally further processed, and used individually or in combination in a variety of applications as described herein.

In one aspect the ECM and CCM are produced under one, two or three dimensional conditions. The three-dimensional support or scaffold used to culture stromal cells may be of any material and/or shape that: (a) allows cells to attach to it (or can be modified to allow cells to attach to it); and (b) allows cells to grow in more than one layer (i.e., form a three dimensional tissue). In other embodiments, a substantially two-dimensional sheet or membrane or beads may be used to culture cells that are sufficiently three dimensional in form.

In one aspect, mesh is used for production of ECM. The mesh is a woven nylon 6 material in a plain weave form with approximately 100 μm openings and approximately 125 μm thick. In culture, fibroblast cells attach to the nylon through charged protein interactions and grow into the voids of the mesh while producing and depositing ECM proteins. Mesh openings that are excessively large or small may not be effective but could differ from those above without substantially altering the ability to produce or deposit ECM. In another aspect, other woven materials are used for ECM production, such as polyolefin's, in weave configurations giving adequate geometry for cell growth and ECM deposition.

For example, nylon mesh is prepared for cultivation in any of the steps of the invention by cutting to the desired size, washing with 0.1-0.5M acetic acid followed by rinsing with high purity water and then steam sterilized. For use as a three-dimensional scaffold for ECM production the mesh is sized into squares approximately 10 cm×10 cm. However, the mesh could be any size appropriate to the intended application and may be used in any of the methods of the present invention, including cultivation methods for inoculation, cell growth and ECM production and preparation of the final form.

In other aspects, the scaffold for generating the cultured tissues is composed of microcarriers, which are beads or particles. The beads may be microscopic or macroscopic and may further be dimensioned so as to permit penetration into tissues or compacted to form a particular geometry. In some tissue penetrating embodiments, the framework for the cell cultures comprises particles that, in combination with the cells, form a three dimensional tissue. The cells attach to the particles and to each other to form a three dimensional tissue. The complex of the particles and cells is of sufficient size to be administered into tissues or organs, such as by injection or catheter. Beads or microcarriers are typically considered a two-dimensional system or scaffold.

As used herein, a “microcarriers” refers to a particle having size of nanometers to micrometers, where the particles may be any shape or geometry, being irregular, non-spherical, spherical, or ellipsoid.

The size of the microcarriers suitable for the purposes herein can be of any size suitable for the particular application. In some embodiments, the size of microcarriers suitable for the three dimensional tissues may be those administrable by injection. In some embodiments, the microcarriers have a particle size range of at least about 1 μm, at least about 10 μm, at least about 25 μm, at least about 50 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1000 μm.

In some aspects in which the microcarriers are made of biodegradable materials. In some aspects, microcarriers comprising two or more layers of different biodegradable polymers may be used. In some embodiments, at least an outer first layer has biodegradable properties for forming the three dimensional tissues in culture, while at least a biodegradable inner second layer, with properties different from the first layer, is made to erode when administered into a tissue or organ.

In some aspects, the microcarriers are porous microcarriers. Porous microcarriers refer to microcarriers having interstices through which molecules may diffuse in or out from the microparticle. In other embodiments, the microcarriers are non-porous microcarriers. A nonporous microparticle refers to a microparticle in which molecules of a select size do not diffuse in or out of the microparticle.

Microcarriers for use in the compositions are biocompatible and have low or no toxicity to cells. Suitable microcarriers may be chosen depending on the tissue to be treated, type of damage to be treated, the length of treatment desired, longevity of the cell culture in vivo, and time required to form the three dimensional tissues. The microcarriers may comprise various polymers, natural or synthetic, charged (i.e., anionic or cationic) or uncharged, biodegradable, or nonbiodegradable. The polymers may be homopolymers, random copolymers, block copolymers, graft copolymers, and branched polymers.

In some aspects, the microcarriers comprise non-biodegradable microcarriers. Non-biodegradable microcapsules and microcarriers include, but not limited to, those made of polysulfones, poly(acrylonitrile-co-vinyl chloride), ethylene-vinyl acetate, hydroxyethylmethacrylate-methyl-methacrylate copolymers. These are useful to provide tissue bulking properties or in embodiments where the microcarriers are eliminated by the body.

In some aspects, the microcarriers comprise degradable scaffolds. These include microcarriers made from naturally occurring polymers, non-limiting example of which include, among others, fibrin, casein, serum albumin, collagen, gelatin, lecithin, chitosan, alginate or poly-amino acids such as poly-lysine. In other aspects, the degradable microcarriers are made of synthetic polymers, non-limiting examples of which include, among others, polylactide (PLA), polyglycolide (PGA), poly(lactide-co-glycolide) (PLGA), poly(caprolactone), polydioxanone trimethylene carbonate, polyhybroxyalkonates (e.g., poly(hydroxybutyrate), poly(ethyl glutamate), poly(DTH iminocarbony(bisphenol A iminocarbonate), poly(ortho ester), and polycyanoacrylates.

In some aspects, the microcarriers comprise hydrogels, which are typically hydrophilic polymer networks filled with water. Hydrogels have the advantage of selective trigger of polymer swelling. Depending on the composition of the polymer network, swelling of the microparticle may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide); poly(N-isopropylacrylamide); poly(methacrylic acid-g-polyethylene glycol); polyacrylic acid and poly(oxypropylene-co-oxyethylene) glycol; and natural compounds such as chrondroitan sulfate, chitosan, gelatin, fibrinogen, or mixtures of synthetic and natural polymers, for example chitosan-poly (ethylene oxide). The polymers may be crosslinked reversibly or irreversibly to form gels adaptable for forming three dimensional tissues.

In exemplary aspects, the microcarriers or beads for use in the present invention are composed wholly or composed partly of dextran.

In accordance with the present invention the culturing method is applicable to proliferation of different types of cells, including stromal cells, such as fibroblasts, and particularly primary human neonatal foreskin fibroblasts. In various aspects, the cells inoculated onto the scaffold or framework can be stromal cells comprising fibroblasts, with or without other cells, as further described below. In some embodiments, the cells are stromal cells that are typically derived from connective tissue, including, but not limited to: (1) bone; (2) loose connective tissue, including collagen and elastin; (3) the fibrous connective tissue that forms ligaments and tendons, (4) cartilage; (5) the ECM of blood; (6) adipose tissue, which comprises adipocytes; and (7) fibroblasts.

Stromal cells can be derived from various tissues or organs, such as skin, heart, blood vessels, bone marrow, skeletal muscle, liver, pancreas, brain, foreskin, which can be obtained by biopsy (where appropriate) or upon autopsy. In one aspect, fetal fibroblasts can be obtained in high quantity from foreskin, such as neonatal foreskins.

A discussed throughout, the compositions of the present invention includes both soluble (CCM) and non-soluble fractions (ECM) or any portion thereof. It is to be understood that the compositions of the present invention may include either or both fractions, as well as any combination thereof. Additionally, individual components may be isolated from the fractions to be used individually or in combination with other isolates or known compositions. Such compositions can be produced under normoxic or hypoxic conditions when CCM or ECM is desired for the composition.

In one aspect, the cells are incubated under hypoxic conditions. Incubation of the inoculated culture maybe performed under hypoxic conditions, which is discovered to produce an ECM and CCM with unique properties as compared to ECM and CCM generated under normal culture conditions. As used herein, hypoxic conditions are characterized by a lower oxygen concentration as compared to the oxygen concentration of ambient air (approximately 15%-20% oxygen). In one aspect, hypoxic conditions are characterized by an oxygen concentration less than about 10%. In another aspect hypoxic conditions are characterized by an oxygen concentration of about 1% to 10%, 1% to 9%, 1% to 8%, 1% to 7%, 1% to 6%, 1% to 5%, 1% to 4%, 1% to 3%, or 1% to 2%. In a certain aspect, the system maintains about 1-3% oxygen within the culture vessel. Hypoxic conditions can be created and maintained by using a culture apparatus that allows one to control ambient gas concentrations, for example, an anaerobic chamber.

Incubation of cell cultures is typically performed in normal atmosphere with 15-20% oxygen and 5% CO₂ for expansion and seeding, at which point low oxygen cultures are split to an airtight chamber that is flooded with 95% nitrogen/5% CO₂ so that a hypoxic environment is created within the culture medium.

The division, differentiation, and function of stem cells and multipotent progenitors are influenced by complex signals in the microenvironment, including oxygen availability. Regions of severe oxygen deprivation (hypoxia) arise in tumors for example due to rapid cell division and aberrant blood vessel formation. The hypoxia-inducible factors (HIFs) mediate transcriptional responses to localized hypoxia in normal tissues and in cancers and can promote tumor progression by altering cellular metabolism and stimulating angiogenesis. Recently, HIFs have been shown to activate specific signaling pathways such as Notch and the expression of transcription factors such as Oct4 that control stem cell self-renewal and multipotency. As many cancers are thought to develop from a small number of transformed, self-renewing, and multipotent “cancer stem cells,” these results suggest new roles for HIFs in tumor progression. The data shown in the present examples indicate that the cells cultured under hypoxic conditions express genes typically associated with pluripotent cells, such as Oct4, NANOG, Sox2, KLF4 and cMyc, for example.

The invention is based in part, on the discovery that cells cultured on beads or three-dimensional surfaces under conditions that stimulate the early embryonic environment (hypoxia and reduced gravitational forces) prior to angiogenesis produces ECM compositions with fetal properties, including generation of embryonic proteins. Growth of cells under hypoxic conditions demonstrate a unique ECM with fetal properties and growth factor expression and a unique CCM. Unlike the culturing of ECM under traditional culture conditions, over 5000 genes are differentially expressed in ECM cultured under hypoxic conditions. This results in a cultured ECM that has different properties and a different biological composition. For example, an ECM produced under hypoxic conditions is similar to fetal mesenchymal tissue in that it is relatively rich in collagens type III, IV, and V, and glycoproteins such as fibronectin, SPARC, thrombospondin, and hyaluronic acid.

Autophagy is a self-degradative process that is important for balancing sources of energy at critical times in development and in response to nutrient stress. Autophagy also plays a housekeeping role in removing misfolded or aggregated proteins, clearing damaged organelles, such as mitochondria, endoplasmic reticulum and peroxisomes, as well as eliminating intracellular pathogens. Thus, autophagy is generally thought of as a survival mechanism, although its deregulation has been linked to non-apoptotic cell death. Autophagy can be either non-selective or selective in the removal of specific organelles, ribosomes and protein aggregates, although the mechanisms regulating aspects of selective autophagy are not fully worked out. In addition to elimination of intracellular aggregates and damaged organelles, autophagy promotes cellular senescence and cell surface antigen presentation, protects against genome instability and prevents necrosis.

The proteasome is a protein complex in cells containing proteases; it breaks down proteins that have been tagged by ubiquitin. Proteasomes are part of a major mechanism by which cells regulate the concentration of particular proteins and degrade misfolded proteins. Proteins are tagged for degradation with a small protein called ubiquitin. The tagging reaction is catalyzed by enzymes called ubiquitin ligases. Once a protein is tagged with a single ubiquitin molecule, this is a signal to other ligases to attach additional ubiquitin molecules. The result is a polyubiquitin chain that is bound by the proteasome, allowing it to degrade the tagged protein. The degradation process yields peptides of about seven to eight amino acids long, which can then be further degraded into shorter amino acid sequences and used in synthesizing new proteins. The proteasome most exclusively used in mammals is the cytosolic 26S proteasome, which is about 2000 kilodaltons (kDa) in molecular mass containing one 20S protein subunit and two 19S regulatory cap subunits. The core is hollow and provides an enclosed cavity in which proteins are degraded; openings at the two ends of the core allow the target protein to enter. Each end of the core particle associates with a 19S regulatory subunit that contains multiple ATPase active sites and ubiquitin binding sites; it is this structure that recognizes polyubiquitinated proteins and transfers them to the catalytic core.

In one aspect, expression of genes and/or polypeptides associated with autophagy and/or proteasome activation are increased following administration of the CCM composition. Genes involved in autophagy and/or proteasome activation include ATG5, ATG7, ATG12, BECN1, MAP1LC3, POMP, PSMB5, PSMB6 or a combination thereof. Polypeptides involved in autophagy and/or proteasome activation include Autophagy related 5, Autophagy related 7, Autophagy related 12, Beclin-1, Microtubule-associated proteins 1A/1B light chain 3, Proteasome maturation protein, Proteasome subunit beta type-5, Proteasome subunit beta type-5 or a combination thereof. The expression level of genes and/or polypeptides associated with autophagy may increase following the administration of the CCM composition locally where the CCM was administered or systemically in the subject. Increased levels of genes and/or polypeptides may be determined using any known methods in the art such as microarrays, PCR and flow cytometry.

Autophagy related 5 (ATG5) is a protein that, in humans, is encoded by the ATG5 gene located on Chromosome 6. It is an E3 ubi autophagic cell death. ATG5 is a key protein involved in the extension of the phagophoric membrane in autophagic vesicles. It is activated by ATG7 and forms a complex with ATG12 and ATG16L1. This complex is necessary for LC3-I (microtubule-associated proteins 1A/1B light chain 3B) conjugation to PE (phosphatidylethanolamine) to form LC3-II (LC3-phosphatidylethanolamine conjugate). ATG5 can also act as a pro-apoptotic molecule targeted to the mitochondria. Under low levels of DNA damage, ATG5 can translocate to the nucleus and interact with survivin. The ATG12-ATG5:ATG16L complex is responsible for elongation of the phagophore in the autophagy pathway. ATG12 is first activated by ATG7, proceeded by the conjugation of ATG5 to the complex by ATG10 via a ubiquitination-like enzymatic process. The ATG12-ATG5 then forms a homo-oligomeric complex with ATG16L. With the help of ATG7 and ATG3, the ATG12-ATG5:ATG16L complex conjugates the C terminus of LC3-I to phosphatidylethanolamine in the phospholipid bilayer, allowing LC3 to associate with the membranes of the phagophore, becoming LC3-II. After formation of the autophagosome, the ATG12-ATG5:ATG16L complex dissociates from the autophagosome.

Autophagy related 7 is a protein in humans encoded by ATG7 gene. Related to GSA7; APG7L; APG7-LIKE. ATG 7, present in both plant and animal genomes, acts as an essential protein for cell degradation and its recycling. The sequence associates with the ubiquitin- proteasome system, UPS, required for the unique development of an autophagosomal membrane and fusion within cells. ATG7 was identified based on homology to yeast cells Pichia pastoris GSA7 and Saccharomyces cerevisiae APG7. The protein appears to be required for fusion of peroxisomal and vacuolar membranes. Autophagy is an important cellular process that helps in maintaining homeostasis. It goes through destroying and recycling the cytoplasmic organelles and macromolecules. During the initiation of autophagy, ATG7 acts like an E-1 enzyme for ubiquitin-like proteins (UBL) such as ATG12 and ATG8. ATG7 helps these UBL proteins in targeting their molecule by binding to them and activating their transfer to an E-2 enzyme. ATG7's role in both of these autophagy-specific UBL systems makes it an essential regulator of autophagosome assembly. Homologous to the ATP-binding and catalytic sites of E1 activator proteins, ATG7 uses its cysteine residue to create a thiol-ester bond with free Ubiquitin molecules. Through UPS, Ubiquitin will continue to bind to other autophagy-related proteins, E2 conjugation proteins and E3 protein ligases, to attach Ubiquitins to a target substrate to induce autophagy. ATG7 is often associated with ATG12/ATG5 sequenced ubiquitination cascade. As well in presence of p53 cell cycle pathways during stressed and nutrient poor environments.

Autophagy related 12 is a protein that in humans is encoded by the ATG12 gene. Autophagy is a process of bulk protein degradation in which cytoplasmic components, including organelles, are enclosed in double-membrane structures called autophagosomes and delivered to lysosomes or vacuoles for degradation. ATG12 is the human homolog of a yeast protein involved in autophagy. Autophagy requires the covalent attachment of the protein Atg12 to ATG5 through a ubiquitin-like conjugation system. The Atg12-Atg5 conjugate then promotes the conjugation of ATG8 to the lipid phosphatidylethanolamine.

Beclin-1 is a protein that in humans is encoded by the BECN1 gene. Beclin-1 is a mammalian ortholog of the yeast autophagy-related gene 6 (Atg6) and BEC-1 in the C. elegans nematode. This protein interacts with either BCL-2 or PI3k class III, playing a critical role in the regulation of both autophagy and cell death.

Microtubule-associated proteins 1A/1B light chain 3 (hereafter referred to as LC3) is a protein that in humans is encoded by the MAP1LC3 gene. LC3 is a central protein in the autophagy pathway where it functions in autophagy substrate selection and autophagosome biogenesis. LC3 is the most widely used marker of autophagosomes.

Proteasome maturation protein is a protein that in humans is encoded by the POMP gene. It is a short-lived maturation factor required for 20S proteasome subunit biogenesis.

Proteasome subunit beta type-5 as known as 20S proteasome subunit beta-5 is a protein that in humans is encoded by the PSMB5 gene. This protein is one of the 17 essential subunits (alpha subunits 1-7, constitutive beta subunits 1-7, and inducible subunits including beta1i, beta2i, beta5i) that contributes to the complete assembly of 20S proteasome complex. In particular, proteasome subunit beta type-5, along with other beta subunits, assemble into two heptameric rings and subsequently a proteolytic chamber for substrate degradation. This protein contains “chymotrypsin-like” activity and is capable of cleaving after large hydrophobic residues of peptide. The eukaryotic proteasome recognized degradable proteins, including damaged proteins for protein quality control purpose or key regulatory protein components for dynamic biological processes. An essential function of a modified proteasome, the immunoproteasome, is the processing of class I MHC peptides.

Proteasome subunit beta type-6 also known as 20S proteasome subunit beta-1 is a protein that in humans is encoded by the PSMB6 gene. This protein is one of the 17 essential subunits (alpha subunits 1-7, constitutive beta subunits 1-7, and inducible subunits including beta1i, beta2i, beta5i) that contributes to the complete assembly of 20S proteasome complex. In particular, proteasome subunit beta type-6, along with other beta subunits, assemble into two heptameric rings and subsequently a proteolytic chamber for substrate degradation. This protein contains “Caspase-like” activity and is capable of cleaving after acidic residues of peptide. The eukaryotic proteasome recognized degradable proteins, including damaged proteins for protein quality control purpose or key regulatory protein components for dynamic biological processes. An essential function of a modified proteasome, the immunoproteasome, is the processing of class I MHC peptides.

Accordingly, in various aspects, the compositions produced using the methods of the present invention may be used directly or processed in various ways, the methods of which may be applicable to both the ECM and CCM compositions. The CCM, including the cell-free supernatant and media, may be subject to lyophilization for preserving and/or concentrating the factors. Various biocompatible preservatives, cryoprotectives, and stabilizer agents may be used to preserve activity where required. Examples of biocompatible agents include, among others, glycerol, dimethyl sulfoxide, and trehalose. The lyophilizate may also have one or more excipients such as buffers, bulking agents, and tonicity modifiers. The freeze-dried media may be reconstituted by addition of a suitable solution or pharmaceutical diluent, as further described below.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, alveolar lavage, intracranial as well infusion, inhalation, and nebulization. The phrases “parenteral administration” and “administered parenterally” as used herein means modes of administration other than enteral and topical administration.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The actual final dosage for a given route of administration is easily determined by routine experimentation. Depending on the condition being treated, these pharmaceutical compositions may be formulated and administered systemically or locally. Techniques for formulation and administration are generally known in the art. Suitable routes may, for example, parenteral delivery, including intramuscular, subcutaneous, intramedullary, intrathecal, intraventricular, intravenous, or intraperitoneal. For injection, the pharmaceutical compositions of the invention may be formulated in aqueous solutions, preferably in physiologically compatible buffers such as Hanks' solution, Ringer's solution, or physiologically buffered saline.

In an aspect, the CCM composition maybe administered in a nano-container conjugated to a targeting moiety for tissue specific delivery. Nano-containers for targeted drug delivery. Nano-containers have been developed for smart delivery of drugs to the desired organs or tissues, which reduces the risk of side effects. The nano-containers protect the cargo from external influence/environment and release it only in response to certain environmental conditions at the desired destination, for example for release of cargo in various pH mediums. The targeting moiety specifically binds a target tissue and maybe an antibody, antibody fragment, scFv, or Fc-containing polypeptide. In another aspect, a nano-container is a nanosized vessel, which contains the active substances, i.e. the CCM composition, in its interior (hollow structure) or in inner cavities (porous structure).

In one aspect the CCM is administered as a pharmaceutical composition.

As used herein, “pharmaceutical composition” refers to a formulation comprising an active ingredient, and optionally a pharmaceutically acceptable carrier, diluent or excipient. The term “active ingredient” can interchangeably refer to an “effective ingredient”, and is meant to refer to any agent that is capable of inducing a sought-after effect upon administration. Examples of active ingredient include, but are not limited to, chemical compound, drug, therapeutic agent, small molecule, etc. In one aspect, the atcive ingredient is the CCM composition.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

In an additional embodiment, the present invention provides a method of reducing symptoms of aging in a subject comprising administering to the subject a cell culture conditioned medium (CCM) composition. In one aspect, the CCM composition is produced by culturing cells in a suitable growth medium, wherein the cells produce and secrete a cell culture conditioned medium (CCM) composition. In an additional aspect, the symptoms of aging are selected from the group consisting of fine lines, wrinkles, dark spots, dry skin, loosening of the skin and dulling of skin tone. In a further aspect, the CCM composition is administered topically.

As human beings age, there are noticeable changes in the condition of the skin. Symptoms of aging include: loosening of the skin as there is loss of the elastic tissue (elastin); skin becomes thinner, fine lines and wrinkles appear; dark spots appear; and skin becomes dry.

In a further aspect, the present invention provides a method of preventing, treating and/or ameliorating symptoms of a disease or condition in a subject comprising administering to the subject a cell culture conditioned medium (CCM) composition. In one aspect, the CCM composition is produced by culturing cells in a suitable growth medium, wherein the cells produce and secrete a cell culture conditioned medium (CCM) composition. In another aspect, the disease or condition is Alzheimer's disease, lung fibrosis, Huntington's disease, Amyotrophic lateral sclerosis, muscular dystrophies characterized by downregulated proteasome functions and other diseases of aging caused by diminished autophagy and proteasome activity. In a further aspect, the CCM composition is administered by topical, oral, nasal, alveolar lavage, inhalation, intravenous or intracranial administration.

Alzheimer's disease is a chronic neurodegenerative disease that usually starts slowly and gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioral issues.

Lung fibrosis or Pulmonary fibrosis is a respiratory disease in which scars are formed in the lung tissues, leading to serious breathing problems. Scar formation, the accumulation of excess fibrous connective tissue (the process called fibrosis), leads to thickening of the walls, and causes reduced oxygen supply in the blood. As a consequence patients suffer from perpetual shortness of breath.

Inclusion body myopathy (IBM) is a form of muscular dystrophy that can have inflammatory, auto-immune and genetic origins. Some of the genetically caused IBMs, such as hereditary inclusion body myopathy with Paget's disease of bone and frontotemporal dementia (hIBMPDFTD) are caused by a malfunction of a ubiqutylated protein carrier complex, VCP/p97 caused by genetic mutations that affect the processivity of the enzyme to deliver ubiquitylated proteins to the proteasome. (16) While in young people these mutations are silent, the proteasome function loss connected with aging in conjunction with slowed delivery of protein substrates intended for degradation will lead to a backlog, overwhelming the protein removal mechanism and result in the formation of inclusion bodies and ultimately cell death in tissues, such as muscle, bone and neurons, which generally require a high protein turnover.

Huntington's Disease is the most prominent version of hereditary poly-Q diseases. It is characterized by progressive muscle weakness, cognitive decline and psychosis. (17) It is caused by the aberrant poly-Q form of huntingtin forming aggregates, which normally can be degraded by juvenile and middle-aged proteasomes, but which accumulate in senescent muscle and nerve cells later in age.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

### Differential Gene Expression of Skin Treated with a CCM

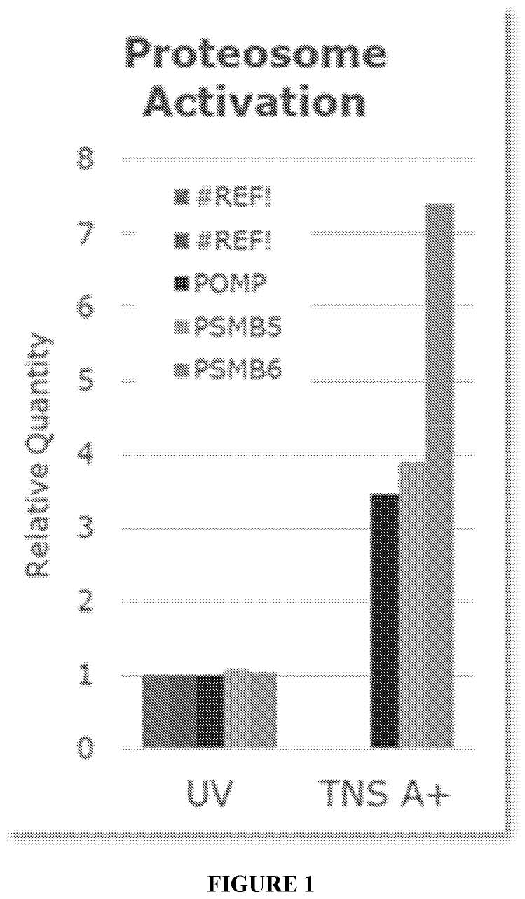

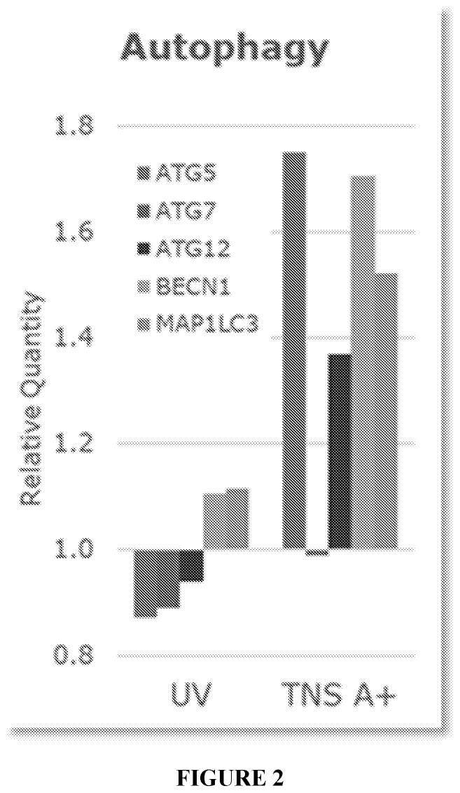

A human 3D skin model was treated with UV light to simulate extrinsic gaining changes. The model was treated with a CCM composition post UV exposure and the expression of genes associated with autophagy and proteasome activation were measured following administration. FIG. 1 shows that genes related to proteasome activation (POMP, PSMB5 and PSMB6) are increased in the skin model treated with the CCM. FIG. 2 shows that genes associated with autophagy (AT5, ATG7, ATG12, BECN1 and MAP1LC3) are increased in the skin model treated with the CCM.

Although the invention has been described with reference to the above examples, it will be understood that modifications and variations are encompassed within the spirit and scope of the invention. Accordingly, the invention is limited only by the following claims.

What is claimed:

1. A method of promoting autophagy and/or proteasome activation in a subject comprising administering to the subject a cell culture conditioned medium (CCM) composition, thereby promoting autophagy and/or proteasome activation.

2. The method of claim 1, wherein the CCM composition is produced by a method comprising:
culturing cells in a suitable growth medium, wherein the cells produce and secrete a cell culture conditioned medium (CCM) composition.

3. The method of claim 2, wherein the cells are cultured under hypoxic or normoxic conditions.

4. The method of claim 2, wherein the hypoxic conditions comprise 1-5% O₂.

5. The method of claim 2, wherein the cells are fibroblast cells.

6. The method of claim 1, wherein the cells are grown under one, two or three dimensional conditions.

7. The method of claim 6, wherein the cells are grown in a monolayer, on beads or on mesh.

8. The method of claim 1, wherein the CCM composition is administered topical, oral or intravenous administration.

9. The method of claim 1, wherein expression of ATG5, ATG7, ATG12, BECN1, MAP1LC3, POMP, PSMB5, PSMB6 or a combination thereof is increased following administration of the CCM.

10. A method of reducing symptoms of aging in a subject comprising administering to the subject a cell culture conditioned medium (CCM) composition, thereby reducing the symptoms of aging.

11. The method of claim 10, wherein the CCM composition is produced by a method comprising:
culturing cells in a suitable growth medium, wherein the cells produce and secrete a cell culture conditioned medium (CCM) composition.

12. The method of claim 10, wherein symptoms of aging are selected from the group consisting of fine lines, wrinkles, dark spots, dry skin, loosening of the skin, and dulling of skin tone.

13. The method of claim 10, wherein the CCM composition is administered topically.

14. A method of preventing, treating and/or ameliorating symptoms of a disease or condition in a subject comprising administering to the subject a cell culture conditioned medium (CCM) composition, thereby preventing, treating and/or ameliorating symptoms of a disease or condition.

15. The method of claim 14, wherein the CCM composition is produced by a method comprising:
culturing cells in a suitable growth medium, wherein the cells produce and secrete a cell culture conditioned medium (CCM) composition.

16. The method of claim 14, wherein the disease or condition is Alzheimer's disease, lung fibrosis, Huntington's disease, Amyotrophic lateral sclerosis, muscular dystrophies characterized by downregulated proteasome functions and other diseases of aging caused by diminished autophagy and proteasome activity.

17. The method of claim 14, wherein the goal is to prolong general or specific tissue and organ health or a youthful appearance.

18. The method of claim 8, wherein the CCM composition is administered by topical, oral, nasal, alveolar lavage, inhalation, intravenous or intracranial administration.

19. The method of claim 8, wherein the CCM composition is administered in a nano-container conjugated to a targeting moiety for specific tissue delivery.

20. The method of claim 19, where the targeting moiety for specific tissue delivery is an aptamer, antibody, antibody fragment, peptide or affinity ligand that recognizes a tissue specific surface or internalization biomarker.
